(19) United States
(12) Patent Application Publication
(10) Pub. No.: US 2021/0085686 A1
(43) Pub. Date: Mar. 25, 2021

(54) DELIVERY OF ACTIVE AGENTS USING NANOFIBER WEBS

(71) Applicant: CorMedix Inc., Berkeley Heights, NJ (US)

(72) Inventors: Robert DiLuccio, Viera, FL (US); Randy Milby, Long Valley, NJ (US)

(21) Appl. No.: 17/063,142

(22) Filed: Oct. 5, 2020

Related U.S. Application Data
(63) Continuation-in-part of application No. 16/440,587, filed on Jun. 13, 2019.
Application No. 16/440,587 is a continuation of application No. 15/253,176, filed on Aug. 31, 2016, now abandoned.
(60) Provisional application No. 62/211,912, filed on Aug. 31, 2015.
(60) Provisional application No. 62/910,844, filed on Oct. 4, 2019.

(51) Int. Cl. A61K 31/549; A61K 9/70

(52) U.S. Cl. CPC A61K 31/549; A61K 47/34; A61K 9/70

(57) ABSTRACT

An active agent delivery system comprising a nanofiber web and an active agent carried by the nanofiber web.

### REFERENCE TO PENDING PRIOR PATENT APPLICATIONS

This patent application:

(i) is a continuation-in-part of pending prior U.S. patent application Ser. No. 16/440,587, filed Jun. 13, 2019 by CorMedix Inc. and Robert DiLuccio et al. for DELIVERY OF ACTIVE AGENTS USING NANOFIBER WEBS (Attorney's Docket No. CORMEDIX-11 CON), which patent application, in turn, is a continuation of prior U.S. patent application Ser. No. 15/253,176, filed Aug. 31, 2016 by CorMedix Inc. and Robert DiLuccio et al. for DELIVERY OF ACTIVE AGENTS USING NANOFIBER WEBS application claims benefit of prior U.S. Provisional Patent Application Ser. No. 62/211,912, filed Aug. 31, 2015 by CorMedix Inc. and Robert DiLuccio for ANTIMICROBIAL COMPOSITIONS AND METHODS USING NANOFIBER WEBS (Attorney's Docket No. CORMEDIX-11 PROV); and

(ii) claims benefit of pending prior U.S. Provisional Patent Application Ser. No. 62/910,844, filed Oct. 4, 2019 by CorMedix Inc. and Robert DiLuccio et al. for DELIVERY OF ACTIVE AGENTS USING NANOFIBER WEBS (Attorney's Docket No. CORMEDIX-11 CIP PROV).

The four (4) above-identified patent applications are hereby incorporated herein by reference.

### FIELD OF THE INVENTION

This invention relates generally to the delivery of active agents to a patient, and more particularly to the delivery of active agents to a patient using nanofiber webs.

### BACKGROUND OF THE INVENTION

A biodegradable, sustained-release drug delivery device (DDD) has the benefits of (1) delivering an active agent (e.g., a drug) exactly where it is needed, thereby limiting undesirable side effects for the rest of the body, (2) providing higher concentrations of the active agent at a desired site within the body, (3) providing a longer therapeutic interval, by maintaining the active agent at the desired site, (4) enabling fewer re-treatments, due to the greater efficiency of the active agent delivery device, and (5) reducing the need to remove and replace a “spent” active agent delivery device, due to the greater efficiency of the active agent delivery device.

### SUMMARY OF THE INVENTION

Polymeric nanofibers have been developed which are useful in a variety of medical and other applications, such as filtration devices, medical prostheses, scaffolds for tissue engineering, wound dressings, controlled drug delivery systems, cosmetic skin masks, protective clothing, etc. These polymeric nanofibers can be formed out of any of a variety of different polymers, both biodegradable and non-biodegradable, and derived from synthetic or natural sources.

The present invention discloses (1) the composition of fibrous articles, and (2) methods for using these fibrous articles for the delivery of active agents (e.g., drugs). The fibrous articles, which are preferably formed by electrospinning a polymer solution of biodegradable fiberizable material with, or in conjunction with, active agents such as medicinal agents and bioactive materials (in one preferred form of the invention, an antimicrobial material such as taurolidine). Thus, the present invention provides a composite of nanofibers carrying active agents (which may also be referred to as “actives”). Such nanofibrous composites may be used for a variety of purposes, including use as controlled drug delivery devices, glaucoma implants, tissue engineering scaffolds, wound dressings, reinforcement grafts, corneal shields, orbital blowout reconstructive materials, sinus reconstructive materials, etc. The present invention also comprises the provision and use of novel nanofibrous composites for the controlled delivery of an active agent such as a medicinal agent and for providing treatment for inflammation, infection, trauma, glaucoma, degenerative diseases, etc. The compositions and methods of the present invention are directed towards improving the delivery of active agents (e.g., drugs) to a target area of the body. These delivery compositions comprise nanofiber webs, mats, whiskers, etc. which incorporate an active ingredient, preferably an antimicrobial (such as taurolidine) for delivery into a patient for subsequent contact by a bodily fluid. The active agent (e.g., the antimicrobial taurolidine) is delivered in a controlled manner by placing the nanofiber web at an anatomical site, whereupon contact by bodily fluids causes the active agent carried by the nanofiber to be released in a controlled and longer-lasting manner.

One particular aspect of the present invention is the provision and use of novel compositions comprising a nanofiber web, impregnated with an active ingredient (preferably an antimicrobial such as taurolidine), which are introduced onto or into tissues for contact by bodily fluids.

Another particular aspect of the present invention is the provision of delivering an active agent to an anatomical site by placing or positioning a nanofiber web containing the active agent (preferably an antimicrobial such as taurolidine) onto or into tissues for contact by bodily fluids.

In one preferred form of the invention, the invention comprises the provision and use of an active agent delivery system comprising (i) a non-woven structure formed out of polymeric nanofiber (biodegradable or non-biodegradable), and (ii) an active agent carried by the non-woven structure (of the polymeric nanofiber) and which is to be delivered to the body of a patient and released. In one preferred form of the invention, the non-woven structure comprises a polymeric nanofiber which is configured to become a gel when wet by bodily fluids. And in one preferred form of the invention, the active agent comprises an antimicrobial. And in one particularly preferred form of the invention, the active agent comprises taurolidine. The active agent is embedded (i.e., “impregnated”) in the non-woven structure (of the polymeric nanofiber), or otherwise carried by the non-woven structure, either disposed in openings in the non-woven structure or disposed on the surface of the polymeric nanofibers or incorporated in the side-walls of the polymeric nanofibers. In this way, the active agent is delivered to an anatomical site when the non-woven structure of polymeric nanofibers is delivered to the anatomical site, and the active agent is released from the non-woven structure of polymeric nanofibers when the non-woven structure is wet by bodily fluids.

In one preferred form of the present invention, there is provided an active agent delivery system comprising a nanofiber web and an active agent carried by the nanofiber web.

In another preferred form of the present invention, there is provided a method for delivering an active agent to a patient, the method comprising:

providing an active agent delivery system comprising a nanofiber web and an active agent carried by the nanofiber web; and

positioning the active agent delivery system into or onto the body of a patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic representation of an electrospinning process;

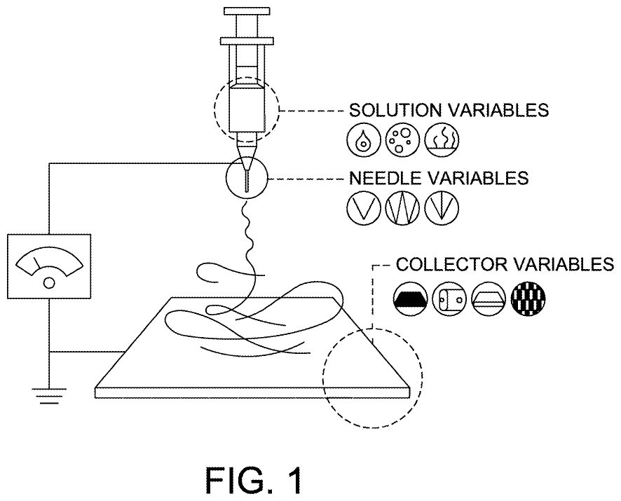

FIG. 2 is a scanning electron micrograph of poly(lactic-co-glycolic acid) (PLGA) nanofibers; and

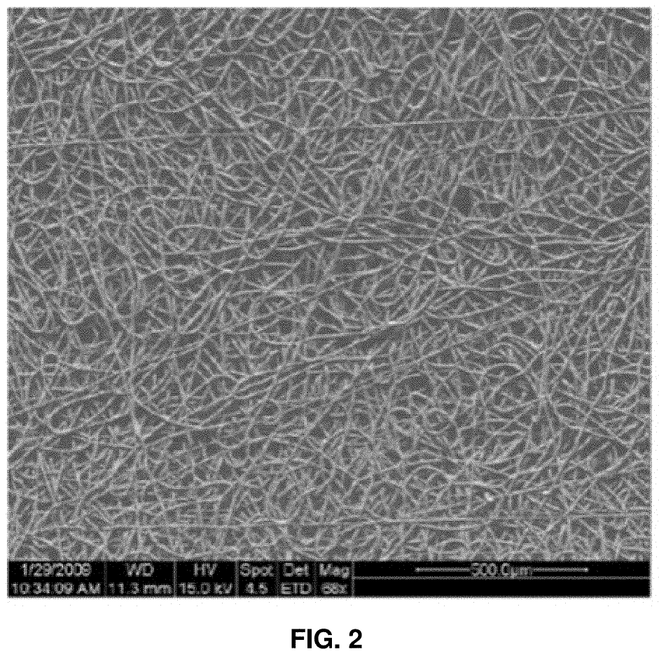

FIG. 3 illustrates zones of inhibition for test samples infused with taurolidine.

### DETAILED DESCRIPTION OF THE PREFERRED EMBODIMENTS

The active agent delivery composition of the present invention preferably comprises a non-woven nanofiber web or mat comprising an active agent or ingredient or ingredients (preferably an antimicrobial such as taurolidine) carried by the non-woven nanofiber web. Preferably the active agent or ingredient (e.g., taurolidine) is dispersed throughout a matrix comprising the nanofiber web, although the invention also provides a nanocomposite wherein the active ingredient is loaded in, or adsorbed to, an article incorporating the nanofiber web (e.g., an in-dwelling catheter incorporating the nanofiber web, a subcutaneous drug port incorporating the nanofiber web, etc.).

More particularly, in one preferred form of the invention, the invention comprises the provision and use of an active agent delivery system comprising (i) a non-woven structure formed out of polymeric nanofiber (biodegradable or non-biodegradable), and (ii) an active agent carried by the non-woven structure (of the polymeric nanofiber) and which is to be delivered to the body of a patient and released. In one preferred form of the invention, the non-woven structure comprises polymeric nanofiber which is configured to become a gel when wet by bodily fluids. And in one preferred form of the invention, the active agent comprises an antimicrobial. And in one particularly preferred form of the invention, the active agent comprises taurolidine. The active agent is embedded (“impregnated”) in the non-woven structure (of the polymeric nanofiber), or otherwise carried by the non-woven structure, either disposed in openings in the non-woven structure or disposed on the surface of the polymeric nanofibers or incorporated in the side-walls of the polymeric nanofibers. In this way, the active agent is delivered to an anatomical site when the non-woven structure of polymeric nanofibers is delivered to the anatomical site, and the active agent is released from the non-woven structure of polymeric nanofibers when the non-woven structure is wet by bodily fluids.

### Nanofiber Web Or Mat

A nanofiber web or mat, for the purposes of the present invention, preferably comprises a non-woven, randomly oriented or aligned collection of nanofibers. These nanofiber webs or mats are typically in the form of a thick and tangled mass defined by an open texture or porosity. For the purposes of the present invention, the terms “nanofiber web”, “nanofiber mat”, “nanofiber mesh” and “nanofiber membrane” may all be used interchangeably (the nanofiber web or mat can also be considered to be something of a membrane—macroscopically, the membrane is a network of nanofibrous structures).

The nanofibers used to form the nanofiber web or mat can be formed from various inorganic, organic, or biological polymers. Preferably these nanofibers are formed by electrospinning. However, other techniques (such as drawing, template synthesis, phase separation or self-assembly) may also be used to produce the nanofibers. All of these techniques are described in “An Introduction to Electrospinning and Nanofibers”, Ramakrishna et al., World Scientific, 2005, which document is hereby incorporated herein by reference. Nanofiber mats or webs can be modified by compression into pellets; by folding into homogeneous or heterogeneous layers; cutting into discs or rings; laminating onto carrier polymers, films, fabrics (woven or non-woven), paper, or biological membranes; or chopped into short segments known as whiskers.

The nanofibers are preferably less than 3 micrometers in diameter, more preferably less than 500 nm in diameter, and most preferably less than 500 nm in diameter and greater than 2 nanometers in diameter.

The thickness of the nanofiber web is preferably less than 10 mm, more preferably less than 5 mm in thickness, and most preferably less than 1 mm in thickness.

Preferably, the polymers used to make the nanofibers of the present invention are biocompatible. For the purposes of the present invention, biocompatibility means the capability of coexistence with living tissues or organisms without causing harm, by not being toxic, injurious, or physiologically reactive, and not causing immunological rejection. The polymers used to make the nanofibers of the present invention can be biodegradable or non-biodegradable and synthetic or natural.

Examples of biocompatible, biodegradable synthetic polymers which may be used with the present invention include, but are not limited to, polyesterurethane (Degrapol™), poly(ϵ-caprolactone), polydioxanone, poly(ethylene oxide), polyglycolide, poly(lactic acid) (PLA), poly(L-lactide-co-ϵ-caprolactone), and poly(lactide-co-glycolide) (PLGA).

Examples of biocompatible non-biodegradable synthetic polymers which may be used with the present invention include, but are not limited to, nylon 4,6; nylon 6; nylon 6,6; nylon 12; polyacrylic acid; polyacrylonitrile; poly(benzimidazol) (PBI); polycarbonate; poly(etherimide) (PEI); poly(ethylene terephthalate); polymethylmethacrylate; polystyrene; polysulfone; poly(urethane); poly(urethane urea); poly(vinyl alcohol); poly(N-vinylcarazole); poly(vinyl chloride); poly(vinyl pyrrolidone); poly(vinylidene fluoride) (PVDF); and hydrogels such as galyfilcon and silicone hydrogels.

Examples of biocompatible natural polymers which may be used with the present invention include, but are not limited to, proteins (collagen, gelatin, fibrinogen, silk, casein, chitosan, etc.) and polysaccharides (cellulose, hyaluronic acid, etc.).

These polymers may be used alone or as co-polymers or laminates with other biodegradable or non-biodegradable polymers. Such non-biodegradable polymers or copolymer blends may be used, for example, as a carrier for drug delivery, for glaucoma surgical adjuncts, orbital/paranasal sinus surgical repair, orbital repair after enucleation, or tissue engineering purposes. It may be necessary to polymerize two different homopolymers to form a copolymer (random or block) or by physical mixing of two or more polymers to form a polymer blend.

As an example, in a preferred embodiment, PLGA is the polymer used to produce the nanofiber web or mat, since it degrades harmlessly to lactic and glycolic acids in vivo, which are then metabolized by cells.

### Active Agent

As disclosed above, the present invention comprises the provision and use of nanofiber webs which carry active agents for controlled release in the body of a patient.

For the purposes of this invention, an “active agent” or “active ingredient” is defined as any material that can be introduced into the body for beneficial effect.

Active agents or ingredients which may be used with the present invention include biological drugs and medicinal agents.

As defined by the National Cancer Institute, a “biological drug” is a substance that is made from a living organism or its products and is used in the prevention, diagnosis, or treatment of cancer and other diseases. Such biological drugs include antibodies, interleukins, growth factors, vaccines, etc. A biological drug may also be called a biologic agent or a biological agent.

For the purposes of the present invention, the term “medicinal agent” is intended to mean any substance, or mixture of substances, which may have any clinical use in medicine. Thus medicinal agents include drugs, enzymes, proteins, peptides, glycoproteins, immunoglobulins, nucleotides, RNA, siRNA, DNA, hormones, and diagnostic agents such as releasable dyes or tracers which may have no biological activity per se but are useful for diagnostic testing (e.g., MRI, etc.).

Examples of classes of medicinal agents that can be used in accordance with the present invention include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunosuppressants, gastrointestinal drugs, diuretics, corticosteroids and enzymes.

It is also intended that combinations of medicinal agents can be used in accordance with the present invention.

Drugs which may be delivered with the present invention include, but are not limited to, many different classes of drugs such as anti-infectives, antibiotics, antituberculosis agents, anti-fungal agents, anti-viral agents, anti-parasitic agents, anti-rheumatic agents, non-steroidal anti-inflammatory drugs (NSAID), corticosteroids, immunomodulators, biologicals, anti-neoplastic agents, etc.

Examples of antibiotics which may be delivered with the present invention include, but are not limited to, aminoglycosides, beta-lactam antibiotics, clindamycin, vancomycin, oxazoladinones, etc. Examples of anti-fungal agents which may be delivered with the present invention include, but are not limited to, amphotericin B and fluconazole, among others. Examples of anti-viral agents which may be delivered with the present invention include, but are not limited to, anti-HIV agents and other antivirals. Examples of anti-parasitic agents which may be delivered with the present invention include, but are not limited to, amebicides and anti-helminthics. Examples of anti-rheumatic agents which may be delivered with the present invention include, but are not limited to, salicylates, e.g., acetylsalicylates and others.

Examples of non-steroidal anti-inflammatory drugs (NSAID) which may be delivered with the present invention include, but are not limited to, acetylsalicylic acid, naproxyn sodium, ibuprofen, diclofenac, indomethacin, cyclooxygenase-2 (COX-2) inhibitors (e.g., rofecoxib) and others. Examples of corticosteroids (glucocorticoids) which may be delivered with the present invention include, but are not limited to, betamethasone, budesonide, cortisone, decadron, dexamethasone, fluocinolone, fluticasone, loteprednol etabonate, methylprednisone, prednisone, prednisolone acetate, prednisolone phosphate, rimexolone, triamcinolone acetonide, immunomodulators, azathioprine, mycophenylate mofetil, cyclophosphamide, cyclosporine A, rapamycin, tacrolimus, methotrexate and others. Examples of biologicals which may be delivered with the present invention include, but are not limited to, anti-bodies such as, tumor necrosis factor (TNF) blockers (such as adalimumab, infliximab and etanercept), daclizumab, aptamers, growth factors, peptides, nucleotides such as DNA, RNA, siRNA and others. Examples of other compounds which may be delivered with the present invention include, but are not limited to, compounds which promote healing and re-endothelialization, e.g., VEGF, Estradiols, antibodies, NO donors, and BCP671. Anti-neoplastic agents (drugs used for treatment of primary central nervous system lymphoma, ocular melanoma and retinoblastoma) may also be delivered with the present invention.

Other preferred medicinal agents include, but are not limited to, corticosteroids, immunomodulators, and biologicals such as aptamers, monoclonal antibodies, and nucleotides. The preferred corticosteroids are budesonide, decadron, dexamethasone, fluocinolone, fluticasone, loteprednol etabonate, methylprednisone, prednisone, prednisolone acetate, prednisolone phosphate, rimexolone and triamcinolone acetonide. The preferred immunomodulators are azathioprine, mycophenylate mofetil, cyclophosphamide, cyclosporine A, rapamycin, tacrolimus, and methotrexate. The preferred monoclonal antibodies are TNF blockers, such as adalimumab, infliximab, etanercept, daclizumab, and anti-VEGF agents such as ranibizumab, bevacizumab, and aptamers.

### Taurolidine

In one preferred form of the present invention, the active agent delivered by the nanofiber webs is taurolidine.

Taurolidine (bis(1,1-dioxoperhydro-1,2,4-thiadiazinyl-4)-methane) is known to have antimicrobial and antilipopolysaccharide properties. Taurolidine is derived from the amino acid taurine. Taurolidine's immunomodulatory actions are reported to be mediated by priming and activation of macrophages and polymorphonuclear leukocytes.

Taurolidine has been used to treat patients with peritonitis and as an antiendoxic agent in patients with systemic inflammatory response syndrome. Taurolidine is a lifesaving antimicrobial for severe abdominal sepsis and peritonitis. For severe surgical infections and use in surgical oncology, taurolidine is active against a wide range of micro-organisms that include gram positive bacteria, gram negative bacteria, fungi and mycobacteria, and also bacteria that are resistant to various antibiotics such as Methicillin-Resistant Staphylococcus Aureus (MRSA), Vancomycin-Intermediate Staphylococcus Aureus (VISA), Vancomycin-Resistant Staphylococcus Aureus (VRSA), Oxacillin-Resistant Staphylococcus Aureus (ORSA), Vancomycin-Resistant Enterococci (VRE), etc. Additionally, taurolidine demonstrates some anti-tumor properties, with positive results seen in early-stage clinical investigations using the drug to treat gastrointestinal malignancies and tumors of the central nervous system.

Taurolidine is the active ingredient of anti-microbial catheter lock solutions for the prevention and treatment of catheter-related blood stream infections (CRBSIs) and is suitable for use in all catheter-based vascular access devices. Bacterial resistance against taurolidine has never been observed in various studies.

Taurolidine acts by a non-selective chemical reaction. In aqueous solution, the parent molecule taurolidine forms an equilibrium with taurultam and N-hydroxymethyl taurultam, with taurinamide being a downstream derivative.

The active moieties of taurolidine are N-methylol derivatives of taurultam and taurinamide, which react with the bacterial cell wall, the cell membrane, and the proteins of the cell membrane, as well as with the primary amino groups of endo- and exotoxins. Microbes are killed and the resulting toxins are inactivated; the destruction time in vitro is 30 minutes.

Pro-inflammatory cytokines and enhanced TNF-α levels are reduced when used as a catheter lock solution.

Taurolidine decreases the adherence of bacteria and fungi to host cells by destructing the fimbriae and flagella and thus prevents the formation of biofilms.

A dose of 5 g of taurolidine, over 2 hours, every 4 hours, for at least 48 hours, was given intravenously for the treatment of various sepsis conditions and beneficial results observed.

### Incorporating The Active Agent Into The Nanofiber Web

The active agent is embedded (i.e., “impregnated”) in the non-woven structure (of the polymeric nanofiber), or otherwise carried by the non-woven structure, either disposed in openings in the non-woven structure or disposed on the surface of the polymeric nanofibers or incorporated in the side-walls of the polymeric nanofibers such that when the non-woven structure is delivered to an anatomical site and exposed to bodily fluids, the active agent is released from the non-woven structure.

In accordance with the present invention, electrospinning or encapsulation techniques may be used to provide for sustained drug release from the polymer nanofiber web.

Historically, PLGA poly(lactide-co-glycolide) has been successfully electrospun with a number of drugs, including tetracycline and ibuprofen, to form absorbable sutures. However, they were solely reliant on compositions of PLGA which were 50:50 poly(lactide-co-lactide) copolymers, which are the easiest copolymer of that composition for creating drug delivery systems, mostly because of their amorphous structure. With the present invention, PLGA compositions outside that composition are now also contemplated (e.g., 14/86 or 10/90 PLGA, which tend to be more crystalline versions of the copolymer). Furthermore, with the present invention, polymers other than PLGA are contemplated. Significantly, with the present invention, other active agents (e.g., taurolidine) are also contemplated. And, with the present invention, nanofiber webs, not absorbable suture, are being formed, which provides the ability to deliver much larger amounts of active agents, and which provides the ability to formulate the nanofiber web to optimize its ability to deliver the active agent without consideration for suture-specific issues (e.g., filament strength, filament stretchability, etc.).

The formulation and characteristics of the active agent/polymer composite is influenced not only by the polymer used to produce the nanofiber web or mat, but also by the type of drug chosen for binding with the nanofiber web. A 20% concentration of ibuprofen in 50:50 poly(lactide-co-glycolide), for example, will have a different release profile from a 20% concentration of corticosterone in the same polymer nanofiber web.

The weight of the active ingredient (preferably an antimicrobial such as taurolidine) in the nanofiber web is preferably less than 80 weight percent of the total weight of the active ingredient and the nanofiber web, more preferably less than 50 weight percent of the total weight of the active ingredient and the nanofiber web, and most preferably less than 20 weight percent of the total weight of the active ingredient and the nanofiber web.

### Active Agent Delivery Using The Nanofiber Webs

Once the active agent has been incorporated into the nanofiber web, the active agent delivery composition of the present invention may be administered in a number of ways. In general the nanofiber web containing the active ingredient is introduced into or onto tissues so that the nanofiber web comes into contact with bodily fluids and the active ingredient is released into the bodily fluids in a controlled manner over a period of time. In the case of a tissue or body fluid, the nanofiber web needs to be positioned or placed in such a manner so as to minimally impair the function of the tissue being treated.

In one embodiment of the present invention, focal delivery and application of a medicinal agent to tissue is achieved. Focal application can be more desirable than general systemic application in many cases, e.g., chemotherapy for localized tumors, because it produces fewer side effects in distant tissues or organs and also concentrates therapy at intended sites. Focal application of growth factors, anti-inflammatory agents, immune system suppressants and/or antimicrobials by the membranes of the present invention is an ideal drug delivery system to speed healing of a wound or incision.

A bodily fluid, for the purposes of this invention, is any fluid found in the body of humans and animals including intra- and extracellular fluids. Examples of these extracellular fluids are subcutaneous fluids, enteral fluids, parenteral fluids, peritoneal fluids, blood, cerebrospinal fluids, glandular fluids (such as pancreatic, hepatic, gallbladder, etc.) plasma, tissue, and other body fluids.

### EXAMPLE

Taurolidine Loaded Poly (d,l LGA) Electrospun Mats. Taurolidine was incorporated in poly(lactide-co-glycolide) 14/86 (Poly d,l LGA, Sigma, MW 66-107 kDa) in order to investigate both the ability of the electrospinning system to encapsulate taurolidine and to model its effectiveness as an antimicrobial delivery system using zone of inhibition (ZOI) testing. Electrospinning Method. Solutions containing taurolidine and polymer were allowed to dissolve overnight at 60° C. prior to electrospinning. Taurolidine-loaded samples were prepared by dissolving the drug into 14/86 Poly (d,l LGA) along with a solvent system, a 1:1 ratio of DMF/THF. Two drug preparations in electrospun fibers were targeted at 0.5% and 1.0% (wt/vol) taurolidine. An unloaded control (no taurolidine) was prepared with poly d,l LGA (14/86). The poly (d,l LGA) was prepared with the solvent system 1:1 DMF/THF. The polymer was allowed to dissolve overnight at room temperature and all the solutions dissolved completely.

For electrospinning, all solutions were loaded in 3 mL luer lock syringes and electrospun at 16 kV with a separation distance of 10 cm and a flow rate of 0.5 mL/hr. A total of 0.2 mL of solution was electrospun and collected on parchment paper. Zone of inhibition samples were prepared from these mats using a 6 mm biopsy punch. Results for this testing are provided below.

Characterization Of Antimicrobial Resistance Of Nanofiber Mats. Antimicrobial behavior of the nanofiber mats were tested using the Kirby-Bauer Disc Diffusion method with S. aureus. S. aureus was grown in Tryptic Soy broth overnight to a concentration of approximately 1.5×10⁸ CFU/mL (equivalent to a 0.5 McFarland standard, or OD625 of 0.08 to 0.13). The next morning, the overnight inoculum was taken out of the incubator and kept at room temperature. The water bath was pre-heated to 48° C. and pre-made top agar was put in it to melt. While the top agar melted, 300 μL of the overnight inoculum was pipetted into test tubes, three for each of the samples that were tested. After the top agar melted, 3 mL was transferred to each of the test tubes. The test tubes containing the solution were vortexed, and the contents poured onto TSA plates (S. aureus). The plates sat at room temperature to dry, and then nanofiber samples and control discs were placed on their respective plates, in triplicate. The plates were then stored in a 5% CO₂ incubator at 37° C. for 24 hours. Results for each experiment are discussed below.

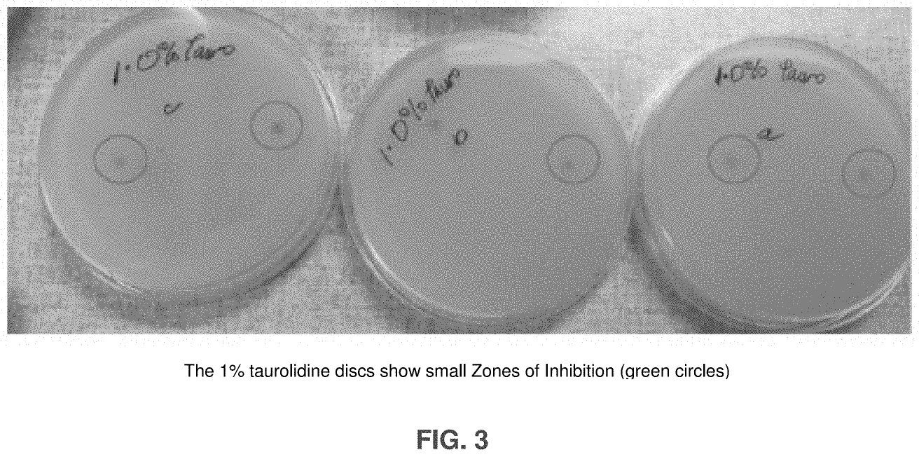

Taurolidine-Loaded Nanofibers. In the first experimental run with two different loadings of taurolidine in Poly (d,1 LGA) nanofiber mats, there was a noticeable zone of inhibition on the plates containing 1.0% taurolidine (FIG. 3, green circles). The 0.5% taurolidine sample did not have a noticeable zone of inhibition.

### Additional Examples

It is important to note that the chemistry of adding taurolidine to a polymer is extremely complicated and yields highly unpredictable results, particularly where the combination of taurolidine and a polymer must be electrospun into a fiber, and the manufacturing process used to produce the fiber must be one which does not deactivate the taurolidine during manufacture, which allows the taurolidine to be released from the polymer at the desired site, and which does not interfere with the active moieties of taurolidine being available at the site.

Through experimentation, it has been found that some polymers cannot be electrospun at all (i.e., the polymers cannot be electrospun so as to form a fiber, regardless of whether the polymers include taurolidine or not); and even where the polymers can be electrospun so as to form a fiber, the addition of taurolidine to the polymers frequently interferes with the electrospinning process and prevents the combination of the polymers and taurolidine from being formed into a fiber. In addition, the manufacturing process must be one which does not deactivate the taurolidine during manufacture, which allows the taurolidine to be released from the polymer at the desired site, and which does not interfere with the active moieties of taurolidine being available at the site.

The following experiments were conducted in order to determine which different types of polymers can be blended with taurolidine, and then electrospun into fibers, and then the antimicrobial properties of those fibers were tested.

In one set of experiments, four different types of polymers were evaluated for blending with taurolidine and then electrospinning into fibers. The four polymers evaluated were:

Capromaxx (95/5) polycaprolactone/glycolide

Lactomaxx (65/35) lactide/glycolide

Glycomaxx (75/25) glycolide/lactide

Dioxomaxx 100 polydioxanone.

The four polymers identified above have very different properties in terms of crystallinity, molecular weight and melt temperature. All of these properties have an influence as to whether they can be successfully blended with taurolidine and then electrospun into fibers.

The properties of the four polymers which were evaluated for blending with taurolidine and then electrospinning into fibers are listed in the following table:

| | Inherent | Melt | |
|---|---|---|---|
| | Viscosity | Temperature | |
| Polymer | (dl/g) | (° C.) | Tg (° C.) |
| Glycomaxx (75/25) | 1.62 | 205 | −8.6 |
| Dioxomaxx (100) | 1.73 | 112 | −7.2 |
| Capromaxx (95/5) | 2.1 | 55.29 | −58 |
| Capromaxx (90/10) | 1.14 | 51 | −58 |
| Lactomaxx (65/35) | 0.62 | Amorphous | 49.1 |

All of the polymers and the taurolidine were dissolved in 1,1,1,3,3,3-hexafluoro-2-propanol (HFP; Oakwood Chemical) for processing into small diameter (SD) and large diameter (LD) fibers. Preliminary testing determined that the maximum solubility of taurolidine in HFP was approximately 218 mg/ml. Thus, for preliminary testing, the taurolidine concentration in the electrospinning solutions was maintained at approximately 100 mg/ml.

In order to keep the spinning conditions consistent for all of the polymers tested, for large diameter fibers, the concentration of polymer ranged from approximately 130 to 360 mg/ml and was adjusted according to the molecular weight of the polymer; and for small diameter fibers, the concentration of polymer ranged from approximately 70 to 180 mg/ml and was also adjusted according to the molecular weight of the polymer so as to keep the viscosity of the spinning solution consistent for all of the polymers that were spun.

After preliminary testing, it was determined that the molecular weight of the Capromaxx CG9010 was too low to be successfully electrospun into fibers. All attempts to electrospin the Capromaxx CG9010 into fibers resulted in the formation of a wax-like, film structure.

Electrospinning parameters were then determined for each solution type. All of the polymer solutions were placed in a syringe on a syringe pump (Model

No. 78-01001, Fisher Scientific) with an 18-gauge blunt needle tip attached to the positive voltage lead of a power supply (Spellman CZE1000R, Spellman High Voltage Electronics Corp.). The polymer solutions were dispensed at a specified rate (4-5 ml/hr), air gap distance between the needle tip and mandrel (12.7 to 15.2 cm) and applied voltage (22 kV). The polymer fibers were collected on a grounded stainless steel rectangular mandrel (200×750×5 mm) rotating at 1250 rpm, and translating 6.5 cm/s over 13 cm.

The results of attempting to electrospin the other polymers (with and without taurolidine) are shown in the tables below (one for large diameter fibers and one for small diameter fibers):

Large Diameter Fibers

| | | Taurolidine Spin |
|---|---|---|
| | Fiber Diameter | Concentration |
| Polymer | (microns) | (mg/ml) |
| Glycomaxx (75/25) | 1.18 +/− 0.33 | 0 |
| Glycomaxx (75/25) | 0.98 +/− 0.42 | 100 |
| Dioxomaxx (100) | 1.21 +/− 0.77 | 0 |
| Dioxomaxx (100) | 2.41 +/− 1.1 | 50 |
| Capromaxx (95/5) | Not processable | 0 |
| Capromaxx (95/5) | Not processable | 100 |
| Lactomaxx (65/35) | 2.11 +/− 0.73 | 0 |
| Lactomaxx (65/35) | 1.45 +/− 0.56 | 100 |

Small Diameter Fibers

| | | Taurolidine Spin |
|---|---|---|
| | Fiber Diameter | Concentration |
| Polymer | (microns) | (mg/ml) |
| Glycomaxx (75/25) | 1.03 +/− 0.52 | 0 |
| Glycomaxx (75/25) | Not processable | 100 |
| Dioxomaxx (100) | 0.97 +/− 0.42 | 0 |
| Dioxomaxx (100) | Not processable | 100 |
| Capromaxx (95/5) | 0.57 +/− 0.36 | 0 |
| Capromaxx (95/5) | Not processable | 100 |
| Lactomaxx (65/35) | 0.07 +/− 0.04 | 0 |
| Lactomaxx (65/35) | 0.51 +/− 0.28 | 100 |

The electrospun taurolidine-polymer fibers were then tested to determine their effectiveness in killing bacteria. The following table and chart illustrates the results of these tests:

The foregoing table and chart demonstrate that, after 24 hours of exposure to the electrospun taurolidine-polymer fibers, no bacteria were left alive. Therefore, the manufacturing process did not deactivate the taurolidine, allowed the taurolidine to be released from the polymer at the desired site, and did not interfere with the active moieties of the taurolidine being available at the site.

### Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. An active agent delivery system comprising a nanofiber web and an active agent carried by the nanofiber web.

2. An active agent delivery system according to claim 1 wherein the nanofiber web comprises a non-woven structure.

3. An active agent delivery system according to claim 2 wherein the non-woven structure has a thickness of less than 10 mm.

4. An active agent delivery system according to claim 2 wherein the non-woven structure has a thickness of less than 5 mm.

5. An active agent delivery system according to claim 2 wherein the non-woven structure has a thickness of less than 1 mm.

6. An active agent delivery system according to claim 2 wherein the nanofiber web comprises polymeric nanofibers.

7. An active agent delivery system according to claim 6 wherein the polymeric nanofibers are biodegradable.

8. An active agent delivery system according to claim 6 wherein the polymeric nanofibers are non-biodegradable.

9. An active agent delivery system according to claim 6 wherein the polymeric nanofibers are configured to become a gel when wet by bodily fluids.

10. An active agent delivery system according to claim 6 wherein the polymeric nanofibers are formed out of a polymer selected from the group consisting of poly-(lactide) (PLA), polycaprolactone, poly-(vinyl alcohol), biodegradable polyester, chitosan, poly-(propylene carbonate), and poly-(lactide-glycolide), poly-e-caprolactone, poly-trimethylene carbonate, poly-glycolide, poly-p-dioxanone.

11. An active agent delivery system according to claim 10 wherein the polymer may be a homopolymer, a copolymer or a multimer.

12. An active agent delivery system according to claim 6 wherein the polymeric nanofibers have a diameter of less than 3 micrometers.

13. An active agent delivery system according to claim 6 wherein the polymeric nanofibers have a diameter of less than 500 nanometers.

14. An active agent delivery system according to claim 6 wherein the polymeric nanofibers have a diameter of greater than 2 nanometers and less than 500 nanometers.

15. An active agent delivery system according to claim 2 wherein the active agent is disposed in openings in the non-woven structure.

16. An active agent delivery system according to claim 6 wherein the active agent is disposed on the surface of the polymeric nanofibers.

17. An active agent delivery system according to claim 6 wherein the active agent is incorporated in the side-walls of the polymeric nanofibers.

18. An active agent delivery system according to claim 1 wherein the active agent comprises an antimicrobial.

19. An active agent delivery system according to claim 18 wherein the antimicrobial comprises taurolidine.

20. A method for delivering an active agent to a patient, the method comprising:
providing an active agent delivery system comprising a nanofiber web and an active agent carried by the nanofiber web; and
positioning the active agent delivery system into or onto the body of a patient.
